SEQUENCE LISTING (1 sequence)

SEQ ID NO: 1 - DNA, 8128 bp, Artificial Sequence
  pCCIBAC vector
gcggccgcaaggggttcgcgtcagcgggtgttggcgggtgtcggggctggcttaactatgcggcatcagagcagattgtactgagagtgcaccatatgcggtgtgaaataccacacagatgcgtaaggagaaaataccgcatcaggcgccattcgccattcagctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttgagtattctatagtctcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagcaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgat

(19) United States
(12) Patent Application Publication
(10) Pub. No.: US 2023/0159940 A1
(43) Pub. Date: May 25, 2023

(54) CULTURE MEDIUM FOR INDUCING INCREASE IN PLASMID COPY NUMBER AND USE THEREOF

(71) Applicant: JIANGSU GENSCRIPT BIOTECH CO., LTD., Zhenjiang, Jiangsu (CN)

(72) Inventors: Fanglong ZHAO, Zhenjiang (CN); Yanqiu MA, Zhenjiang (CN); Man WANG, Zhenjiang (CN); Fan LIU, Zhenjiang (CN); Cheng-hsien WU, Zhenjiang (CN)

(73) Assignee: JIANGSU GENSCRIPT BIOTECH CO., LTD., Zhenjiang, Jiangsu (CN)

(21) Appl. No.: 17/417,802

(22) PCT Filed: Dec. 26, 2019
(86) PCT No.: PCT/CN2019/128523

§ 371 Date: Jun. 24, 2021

(30) Foreign Application Priority Data
Dec. 27, 2018 (CN) 201811610822.6

(51) Int. Cl. C12N 15/70; C12N 1/20

(52) U.S. Cl. CPC C12N 15/70; C12N 1/205; C12N 2800/101; C12N 2500/74; C12N 2500/34; C12R 2001/19

(57) ABSTRACT

The present invention provides a culture medium for inducing an increase in a plasmid copy number and use thereof. The culture medium for inducing an increase in a plasmid copy number of the present invention has a plasmid extraction concentration increased by from 45 to 95% compared with a conventional method for inducing a plasmid copy number, and has a plasmid extraction concentration increased by from 110 to 440% compared with an induction method using a culture medium without glucose and arabinose. The culture medium plays an important role in inducing an increase in a plasmid copy number and achieving high-throughput production.

### CROSS-REFERENCE TO RELATED APPLICATIONS

This application claims priority to Chinese Patent Application No. 201811610822.6, entitled “CULTURE MEDIUM FOR INDUCING INCREASE OF PLASMID COPY NUMBER AND USE THEREOF” filed with the China National Intellectual Property Administration on Dec. 27, 2018, which is incorporated herein by reference in its entirety.

### SEQUENCE LISTING

The instant application contains a Sequence Listing which has been submitted electronically in ASCII format and is hereby incorporated by reference in its entirety. The ASCII copy, created on Dec. 13, 2021, is named “Sequence Listing-20793-0012US00” and is 11,023 bytes in size.

### BACKGROUND

### Technical Field

The present invention relates to the field of biotechnologies, and specifically, to a culture medium for inducing an increase in a plasmid copy number and use thereof.

### Related Art

In the field of gene synthesis, about 10% or more of gene sequences produce toxicities to host Escherichia coli. These toxicities are usually introduced by heterologous genes expressed into proteins by the host to affect the physiological metabolism of the host and further the growth of the host. To resist toxic genes, the host Escherichia coli tends to retain plasmids with gene mutation or gene deletion, resulting in reduced stability of the genes and plasmids. The use of low-copy plasmids in production can effectively reduce expression of the toxic genes, and improve growth viability of the host and stability of genes. However, the low-copy plasmids have a low yield and are difficult to extract, thereby affecting production efficiency. To resolve the problem, it is necessary to rapidly induce the low-copy plasmids to have a high copy number before plasmid extraction, thereby improving the plasmid yield. However, existing methods for inducing an increase in a plasmid copy number require a plurality of operations such as transfer, OD600 quantification, and addition of an inducer, which are relatively complex and have difficulty in meeting requirements of actual high-throughput production.

### SUMMARY

To resolve the problem of cumbersome operations of an existing plasmid copy number induction system, the present invention provides a culture medium for inducing an increase in a plasmid copy number and use thereof, which improves production efficiency while simplifying operations.

An aspect of the present invention provides a culture medium for inducing an increase in a plasmid copy number, including tryptone, a yeast extract, glucose, sodium chloride, and arabinose.

In some implementations, the glucose has a concentration of from 0.1 to 10 g/L, preferably from 1 to 5 g/L, and more preferably from 0.5 to 2 g/L.

In some implementations, the glucose has a concentration of from 0.1 to 10 g/L. In some implementations, the glucose has a concentration of from 1 to 5 g/L. In some implementations, the glucose has a concentration of from 0.5 to 2 g/L. In some implementations, the glucose has a concentration of 0.5 g/L, 0.6 g/L, 0.7 g/L, 0.8 g/L, 0.9 g/L, 1 g/L, 1.1 g/L, 1.2 g/L, 1.3 g/L, 1.4 g/L, 1.5 g/L, 1.6 g/L, 1.7 g/L, 1.8 g/L, 1.9 g/L, or 2 g/L. In some implementations, the glucose has a concentration of 0.5 g/L. In some implementations, the glucose has a concentration of 1 g/L. In some implementations, the glucose has a concentration of 2 g/L. In some implementations, the glucose has a concentration of 10 g/L.

In some other implementations, the arabinose has a concentration of from 0.1 to 10 g/L, preferably from 1 to 5 g/L, and more preferably from 0.3 to 5 g/L.

In some other implementations, the arabinose has a concentration of from 0.1 to 10 g/L. In some implementations, the arabinose has a concentration of from 1 to 5 g/L. In some implementations, the arabinose has a concentration of from 0.3 to 0.75 g/L. In some implementations, the arabinose has a concentration of 0.3 g/L, 0.35 g/L, 0.4 g/L, 0.45 g/L, 0.5 g/L, 0.55 g/L, 0.6 g/L, 0.65 g/L, 0.7 g/L, or 0.75 g/L. In some implementations, the arabinose has a concentration of 0.3 g/L. In some implementations, the arabinose has a concentration of 0.6 g/L. In some implementations, the arabinose has a concentration of 0.75 g/L. In some implementations, the arabinose has a concentration of 1 g/L. In some implementations, the arabinose has a concentration of 2 g/L. In some implementations, the arabinose has a concentration of 5 g/L.

In some other implementations, the tryptone has a concentration of from 1 to 15 g/L.

In some other preferred implementations, the tryptone has a concentration of from 5 to 10 g/L.

In some other implementations, the yeast extract has a concentration of from 1 to 15 g/L.

In some other preferred implementations, the yeast extract has a concentration of from 5 to 10 g/L.

In some other implementations, the sodium chloride has a concentration of from 1 to 15 g/L.

In some other preferred implementations, the sodium chloride has a concentration of from 5 to 10 g/L.

In some more preferred implementations, the culture medium further includes one or more of magnesium chloride, potassium chloride, ferric chloride, or calcium chloride.

In some implementations, the plasmid is a plasmid including a replication origin oriV.

In some implementations, the plasmid is a single-copy plasmid including a replication origin oriV.

In some implementations, the plasmid is a single-copy pCCIBAC plasmid.

Another aspect of the present invention provides a method for inducing an increase in a plasmid copy number, including the following steps:

Step (1): transforming Escherichia coli with a plasmid; and

Step (2): inoculating the Escherichia coli obtained in Step (1) into the culture medium for culturing under conditions suitable for culture.

In some implementations, in Step (1), the plasmid is transformed into competent Escherichia coli.

In some other implementations, in Step (2), the Escherichia coli is preferably EPI300 Escherichia coli or EPI400 Escherichia coli.

In some other implementations, in Step (2), a culture temperature is from 20 to 37° C.

In some other implementations, in Step (2), a culture temperature is from 25 to 37° C.

In some other implementations, in Step (2), a culture time is from 4 to 6 h.

In some specific implementations, in Step (1), a single-copy plasmid including a replication origin oriV is transformed into an Escherichia coli strain, and in Step (2), the Escherichia coli is cultured at a temperature of from 20 to 37° C. in a culture medium including glucose with a concentration of from 1 to 5 g/L, arabinose with a concentration of from 1 to 5 g/L, tryptone with a concentration of from 1 to 15 g/L, a yeast extract with a concentration of from 1 to 15 g/L, and sodium chloride with a concentration of from 1 to 15 g/L.

In some other specific implementations, in Step (1), the pCCIBAC plasmid is transformed into EPI300 Escherichia coli or EPI400 Escherichia coli, and in Step (2), the Escherichia coli is cultured at a temperature of from 25 to 37° C. in a culture medium including glucose with a concentration of from 0.5 to 2 g/L, arabinose with a concentration of from 0.3 to 0.75 g/L, tryptone with a concentration of from 5 to 10 g/L, a yeast extract with a concentration of from 5 to 10 g/L, and NaCl with a concentration of from 5 to 10 g/L.

Still another aspect of the present invention provides use of the culture medium in inducing an increase in a plasmid copy number.

The present invention provides a novel culture medium that can induce an increase in a plasmid copy number and improve the plasmid yield. In addition, when the culture medium is used for inducing an increase in a plasmid copy number, operation steps are simple, a culture time is greatly shortened, and a plurality of operations, such as transfer, OD600 quantification, and addition of an inducer, are not required. When the concentration of glucose is lower than 2 g/L, and the concentration of arabinose is lower than 0.75 g/L, the concentration of plasmid extraction is significantly higher than that of a culture medium with other concentrations of the same components. Especially, when the concentration of glucose is from 0.5 to 2 g/L, and the concentration of arabinose is from 0.3 to 0.75 g/L, compared with a conventional method for inducing a plasmid copy number, the concentration of plasmid extraction is increased by more than 45%, and compared with an induction method using a culture medium without glucose and arabinose, the concentration of plasmid extraction is increased by more than 310%. The culture medium plays an important role in inducing an increase in a plasmid copy number and achieving high-throughput production.

### Explanation of Terms

As used herein, the term “plasmid” refers to a DNA molecule outside a chromosome (or a nucleoid) within organisms such as bacteria, yeasts, and actinomycetes. The plasmid exists in cytoplasm and can replicate independently, so that the plasmid can also maintain a constant copy number in progeny cells and express carried genetic information. The plasmid is a closed circular, double-stranded DNA molecule.

The term “pCCIBAC plasmid” refers to a single-copy bacterial artificial chromosome.

The term “plasmid copy number” refers to the number of plasmids in a genome of a specific organism. Under the control of a plasmid replicon, the plasmid copy number may fluctuate in a narrow range with changes of bacterial culture conditions. When growth conditions are constant, the speed of plasmid proliferation is exactly the same as the speed of host cell proliferation, and the copy number remains unchanged.

The term “single-copy plasmid” means that there is only one specific plasmid in a biological genome.

The term “competent” refers to a special physiological state in which a cell can take up DNA molecules from a surrounding environment and the DNA molecules are not easily broken down by restriction endonucleases in the cell.

The term “glucose” refers to a polyhydroxy aldehyde with the molecular formula C₆H₁₂O₆, which is important in the field of biology and is an energy source for living cells and an intermediate product of metabolism.

The term “arabinose” is also referred to as L(+)-arabinose, pectinose, or the like, with the molecular formula C₅H₁₀O₅, which is a levorotatory monosaccharide.

The term “tryptone” is also referred to as casein tryptone and pancreatic digest of casein, which is a high-quality peptone and is light yellow powder formed through concentration and drying. The tryptone is rich in nitrogen sources, amino acids and the like, can be used to prepare various microbial culture media for culture, isolation, proliferation, and identification of bacteria, and can be used to prepare bacterial biochemical characteristics test culture media such as a sterile test culture medium and an anaerobic bacteria culture medium.

The term “yeast extract” is also referred to as yeast flavor, which is abbreviated as YE according to international usage. Main components of the yeast extract are polypeptides, amino acids, 5′-ribonucleotide, B vitamins, and trace elements. The yeast extract is the most ideal raw material for biological culture media and the main raw material in fermentation industries. The yeast extract has the efficacy 8 times that of yeast, which can greatly increase the production rate of bacterial strains and the yield of fermentation products.

### BRIEF DESCRIPTION OF THE DRAWINGS

FIG. 1 shows a result of plasmid extraction in Example 1, where a horizontal coordinate is a sampling time, and a longitudinal coordinate is a concentration of extracted plasmids;

FIG. 2 shows a result of plasmid extraction in Example 2, where a horizontal coordinate is a sampling time, and a longitudinal coordinate is a concentration of extracted plasmids;

FIG. 3 shows a result of plasmid extraction in Example 3, where a horizontal coordinate is a sampling time, and a longitudinal coordinate is a concentration of extracted plasmids;

FIG. 4 shows a result of plasmid extraction in Example 4, where a horizontal coordinate is a sampling time, and a longitudinal coordinate is a concentration of extracted plasmids;

FIG. 5 shows a result of plasmid extraction in Example 5, where a horizontal coordinate is a sampling time, and a longitudinal coordinate is a concentration of extracted plasmids;

FIG. 6 shows a result of plasmid extraction in Example 6, where a horizontal coordinate is a sampling time, and a longitudinal coordinate is a concentration of extracted plasmids;

FIG. 7 shows a result of plasmid extraction in Comparative Example 1, where a horizontal coordinate is a sampling time, and a longitudinal coordinate is a concentration of extracted plasmids; and

FIG. 8 is a diagram of a pCCIBAC vector.

### DETAILED DESCRIPTION

The present invention is further described below with reference to the specific implementations.

Unless otherwise specified, the experimental methods used in the following examples are all conventional methods.

Unless otherwise specified, the materials and reagents used in the following examples can be commercially available.

### Example 1

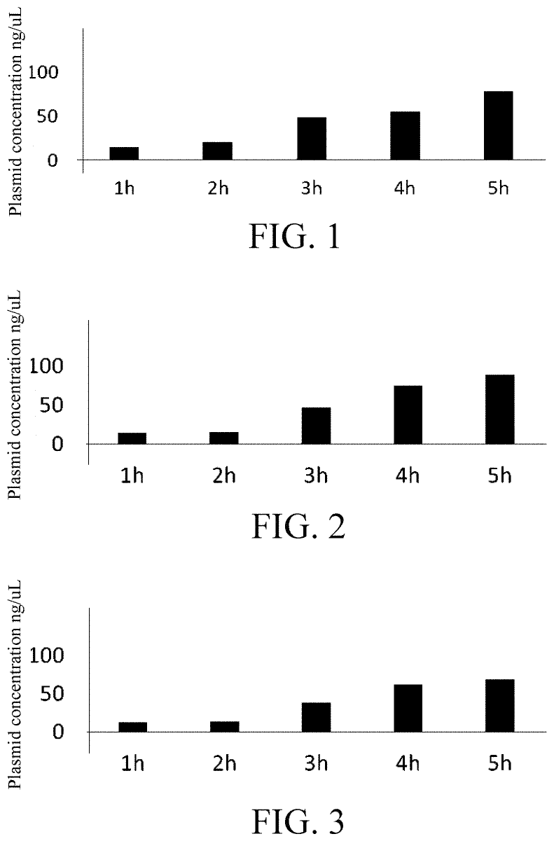

The pCCIBAC plasmid (shown as SEQ ID NO: 1) included a replication origin oriV. The pCCIBAC plasmid was transformed into competent EPI300 Escherichia coli by chemical transformation. The transformed Escherichia coli was cultured in the LB culture medium (in which components were 10 g/L of tryptone, 5 g/L of yeast extract, and 10 g/L of sodium chloride) overnight, and then was inoculated at an inoculation rate of 5% into a culture medium in which components were 10 g/L of tryptone, 5 g/L of yeast extract, 10 g/L of sodium chloride, 2 g/L of glucose, and 0.75 g/L of arabinose. The bacteria were cultured at 220 r/min and 25° C. for 5 h. After the inoculation, 6 OD600 bacteria were taken every 1 h for plasmid extraction. The plasmid extraction was performed by using the standard procedure of the plasmid extraction kit AxyGEN Miniprep Kit (Lot #17718KA1). The result of the plasmid extraction was shown in FIG. 1.

### Example 2

The pCCIBAC plasmid included a replication origin oriV. The plasmid was transformed into competent EPI400 Escherichia coli by chemical transformation. The transformed Escherichia coli was cultured in the LB culture medium (in which components were 10 g/L of tryptone, 5 g/L of yeast extract, and 10 g/L of sodium chloride) overnight, and then was inoculated at an inoculation rate of 5% into a culture medium in which components were 5 g/L of tryptone, 10 g/L of yeast extract, 5 g/L of sodium chloride, 1 g/L of glucose, and 0.6 g/L of arabinose. The bacteria were cultured at 220 r/min and 30° C. for 5 h. After the inoculation, 6 OD600 bacteria were taken every 1 h for plasmid extraction. The plasmid extraction was performed by using the standard procedure of the plasmid extraction kit AxyGEN Miniprep Kit (Lot #17718KA1). The result of the plasmid extraction was shown in FIG. 2.

### Example 3

The pCCIBAC plasmid included a replication origin oriV. The pCCIBAC plasmid was transformed into competent EPI300 Escherichia coli by chemical transformation. The transformed Escherichia coli was cultured in the LB culture medium (in which components were 10 g/L of tryptone, 5 g/L of yeast extract, and 10 g/L of sodium chloride) overnight, and then was inoculated at an inoculation rate of 5% into a culture medium in which components were 10 g/L of tryptone, 10 g/L of yeast extract, 10 g/L of sodium chloride, 0.5 g/L of glucose, and 0.3 g/L of arabinose. The bacteria were cultured at 220 r/min and 37° C. for 5 h. After the inoculation, 6 OD600 bacteria were taken every 1 h for plasmid extraction. The plasmid extraction was performed by using the standard procedure of the plasmid extraction kit AxyGEN Miniprep Kit (Lot #17718KA1). The result of the plasmid extraction was shown in FIG. 3.

### Example 4

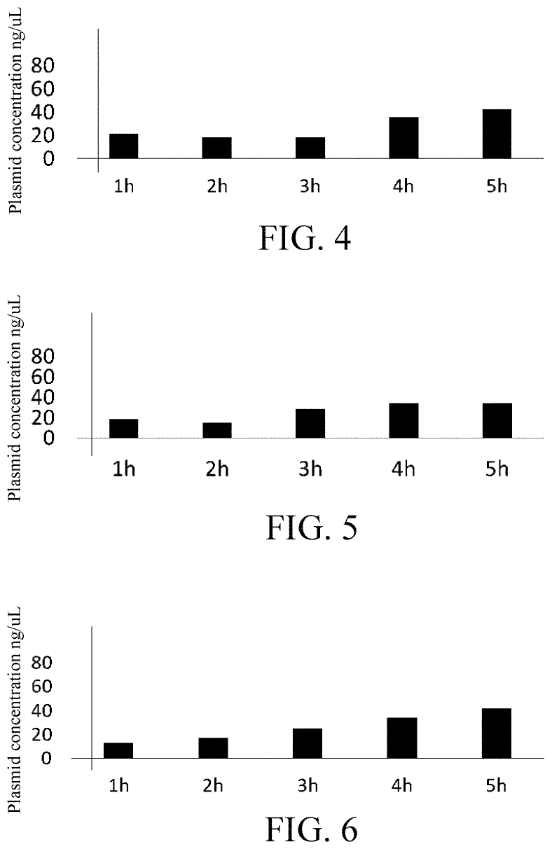

The pCCIBAC plasmid included a replication origin oriV. The plasmid was transformed into competent EPI400 Escherichia coli by chemical transformation. The transformed Escherichia coli was cultured in the LB culture medium (in which components were 10 g/L of tryptone, 5 g/L of yeast extract, and 10 g/L of sodium chloride) overnight, and then was inoculated at an inoculation rate of 5% into a culture medium in which components were 10 g/L of tryptone, 10 g/L of yeast extract, 5 g/L of sodium chloride, 2 g/L of glucose, and 1 g/L of arabinose. The bacteria were cultured at 220 r/min and 30° C. for 5 h. After the inoculation, 6 OD600 bacteria were taken every 1 h for plasmid extraction. The plasmid extraction was performed by using the standard procedure of the plasmid extraction kit AxyGEN Miniprep Kit (Lot #17718KA1). The result of the plasmid extraction was shown in FIG. 4.

### Example 5

The pCCIBAC plasmid included a replication origin oriV. The plasmid was transformed into competent EPI400 Escherichia coli by chemical transformation. The transformed Escherichia coli was cultured in the LB culture medium (in which components were 10 g/L of tryptone, 5 g/L of yeast extract, and 10 g/L of sodium chloride) overnight, and then was inoculated at an inoculation rate of 5% into a culture medium in which components were 10 g/L of tryptone, 10 g/L of yeast extract, 5 g/L of sodium chloride, 10 g/L of glucose, and 5 g/L of arabinose. The bacteria were cultured at 220 r/min and 30° C. for 5 h. After the inoculation, 6 OD600 bacteria were taken every 1 h for plasmid extraction. The plasmid extraction was performed by using the standard procedure of the plasmid extraction kit AxyGEN Miniprep Kit (Lot #17718KA1). The result of the plasmid extraction was shown in FIG. 5.

### Example 6

The pCCIBAC plasmid included a replication origin oriV. The plasmid was transformed into competent EPI400 Escherichia coli by chemical transformation. The transformed Escherichia coli was cultured in the LB culture medium (in which components were 10 g/L of tryptone, 5 g/L of yeast extract, and 10 g/L of sodium chloride) overnight, and then was inoculated at an inoculation rate of 5% into a culture medium in which components were 10 g/L of tryptone, 10 g/L of yeast extract, 5 g/L of sodium chloride, 10 g/L of glucose, and 2 g/L of arabinose. The bacteria were cultured at 220 r/min and 30° C. for 5 h. After the inoculation, 6 OD600 bacteria were taken every 1 h for plasmid extraction. The plasmid extraction was performed by using the standard procedure of the plasmid extraction kit AxyGEN Miniprep Kit (Lot #17718KA1). The result of the plasmid extraction was shown in FIG. 6.

### Comparative Example 1 (without Glucose and Arabinose)

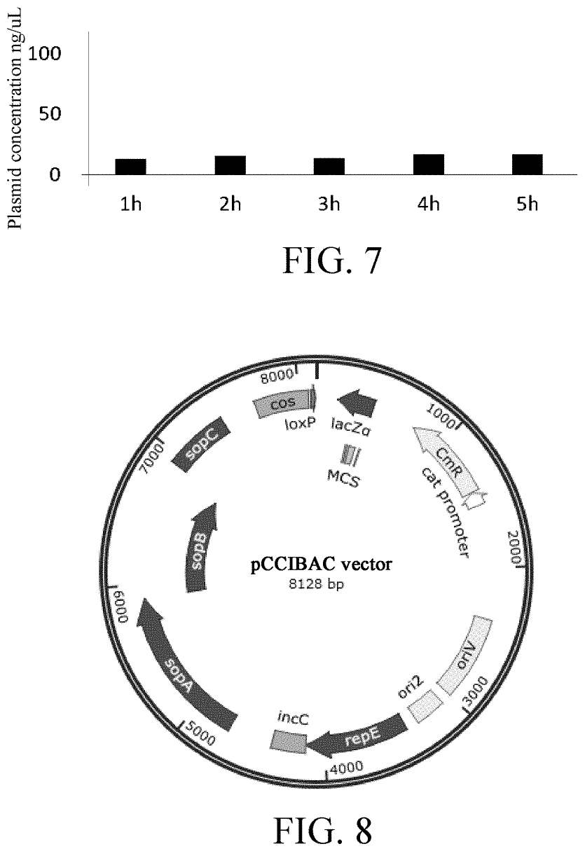

The pCCIBAC plasmid included a replication origin oriV. The pCCIBAC plasmid was transformed into competent EPI300 Escherichia coli by chemical transformation. The transformed Escherichia coli was cultured in the LB culture medium (in which components were 10 g/L of tryptone, 5 g/L of yeast extract, and 10 g/L of sodium chloride) overnight, and then was inoculated at an inoculation rate of 5% into a culture medium in which components were 10 g/L of tryptone, 10 g/L of yeast extract, and 10 g/L of sodium chloride. The bacteria were cultured at 220 r/min and 30° C. After the inoculation, 6 OD600 bacteria were taken every 1 h for plasmid extraction. The plasmid extraction was performed by using the standard procedure of the plasmid extraction kit AxyGEN Miniprep Kit (Lot #17718KA1). The result of the plasmid extraction was shown in FIG. 7.

### Comparative Example 2 (Conventional Method for Inducing a Plasmid Copy Number)

The pCCIBAC plasmid included a replication origin oriV. The pCCIBAC plasmid was transformed into competent EPI300 Escherichia coli by chemical transformation. The transformed Escherichia coli was cultured in the LB culture medium (in which components were 10 g/L of tryptone, 5 g/L of yeast extract, and 10 g/L of sodium chloride) overnight, and then was inoculated at an inoculation rate of 5% into a culture medium in which components were 10 g/L of tryptone, 10 g/L of yeast extract, and 10 g/L of sodium chloride. The bacteria were cultured at 220 r/min and 30° C. for 12 h, and then, were used as a seed solution. Re-inoculation was performed with the seed solution, and OD600 after the re-inoculation was 0.2. Then, the bacteria were cultured at 30° C. for 1 h, and arabinose was added to make the concentration be 0.2 g/L. The culture was continued for 5 h, 6 OD600 bacteria were taken every 1 h for plasmid extraction. The plasmid extraction was performed by using the standard procedure of the plasmid extraction kit AxyGEN Miniprep Kit (Lot #17718KA1). The result of the plasmid extraction was shown in FIG. 8.

After the plasmid extraction, plasmid concentrations were measured by using Nano Drop oneC (Thermo Fisher Scientific). Results were shown in Table 1.

TABLE 1
Plasmid extraction concentration (ng/μL)

| Time | 1 h | 2 h | 3 h | 4 h | 5 h |
|---|---|---|---|---|---|
| Comparative | 12.5 | 15.4 | 13.2 | 16.4 | 16.4 |
| Example 1 | | | | | |
| Example 1 | 14.5 | 20.2 | 48.6 | 55.4 | 78.5 |
| Example 2 | 14.3 | 15.4 | 46.4 | 74.5 | 88.5 |
| Example 3 | 12.8 | 13.3 | 37.8 | 62.3 | 68.4 |
| Example 4 | 21.2 | 18.3 | 18.4 | 35.6 | 42.2 |
| Example 5 | 18.4 | 15.3 | 28.4 | 34.1 | 34.4 |
| Example 6 | 13.1 | 17.4 | 25.2 | 33.8 | 41.5 |
| Comparative | 12.5 | 24.8 | 27.8 | 38.3 | 58.4 |
| Example 2 | | | | | |

The comparison of the plasmid extraction concentrations between Examples 1 to 6 and Comparative Example 1 was shown in Table 2. After glucose and arabinose were added into the culture medium, the concentration of the extracted plasmids increased rapidly. After culture in the culture medium for 5 h, the plasmid extraction concentrations increased by from 110 to 440%. Especially, when the concentration of glucose was from 0.5 to 2 g/L and the concentration of arabinose was from 0.3 to 0.75 g/L, after culture in the culture medium for 5 h, the plasmid extraction concentrations increased by from 317 to 440%. It indicates that the culture medium provided by the present invention is suitable for inducing an increase in a plasmid copy number and greatly improving the plasmid yield.

TABLE 2
Percentages of increases of plasmid extraction concentrations
of Examples 1 to 6 compared with Comparative Example 1

| Time | 1 h | 2 h | 3 h | 4 h | 5 h |
|---|---|---|---|---|---|
| Example 1 | 16% | 31% | 268% | 238% | 379% |
| Example 2 | 14% | 0% | 252% | 354% | 440% |
| Example 3 | 2% | −14% | 186% | 280% | 317% |
| Example 4 | 70% | 19% | 39% | 117% | 157% |
| Example 5 | 47% | −1% | 115% | 108% | 110% |
| Example 6 | 5% | 13% | 91% | 106% | 153% |

Compared with Comparative Example 2 (the conventional method for inducing a plasmid copy number), Examples 1 to 6 had simple operations and greatly improved plasmid extraction concentrations. As shown in Table 3, when the concentration of glucose was from 0.5 to 2 g/L and the concentration of arabinose was from 0.3 to 0.75 g/L, after extraction for 3 h, the plasmid extraction concentrations increased by from 36 to 75% compared to the conventional method, and after extraction for 4 h, the plasmid extraction concentrations increased by from 45 to 95% compared to the conventional method. Especially, when the concentration of glucose was 1 g/L and the concentration of arabinose was 0.6 g/L, after extraction for 4 h, the plasmid extraction concentrations increased by 95% compared to the conventional method.

TABLE 3
Percentages of increases of plasmid extraction concentrations
of Examples 1 to 6 compared with Comparative Example 2

| Time | 1 h | 2 h | 3 h | 4 h | 5 h |
|---|---|---|---|---|---|
| Example 1 | 16% | −19% | 75% | 45% | 34% |
| Example 2 | 14% | −38% | 67% | 95% | 52% |
| Example 3 | 2% | −46% | 36% | 63% | 17% |
| Example 4 | 70% | −26% | −34% | −7% | −28% |
| Example 5 | 47% | −38% | 2% | −11% | −41% |
| Example 6 | 5% | −30% | −9% | −12% | −29% |

SEQ ID NO: 1
pCCIBAC vector sequence
gcggccgcaaggggttcgcgtcagcgggtgttggcgggtgtcggggctggcttaactatgcggcatcagagc
agattgtactgagagtgcaccatatgcggtgtgaaataccacacagatgcgtaaggagaaaataccgcatcag
gcgccattcgccattcagctgcgcaactgttgggaagggcgatcggtgcgggcctcttcgctattacgccagct
ggcgaaagggggatgtgctgcaaggcgattaagttgggtaacgccagggttttcccagtcacgacgttgtaaa
acgacggccagtgaattgtaatacgactcactatagggcgaattcgagctcggtacccggggatcctctagagt
cgacctgcaggcatgcaagcttgagtattctatagtctcacctaaatagcttggcgtaatcatggtcatagctgttt
cctgtgtgaaattgttatccgctcacaattccacacaacatacgagccggaagcataaagtgtaaagcctggggt
gcctaatgagtgagctaactcacattaattgcgttgcgctcactgcccgctttccagtcgggaaacctgtcgtgcc
agctgcattaatgaatcggccaacgcgaaccccttgcggccgcccgggccgtcgaccaattctcatgtttgaca
gcttatcatcgaatttctgccattcatccgcttattatcacttattcaggcgtagcaaccaggcgtttaagggcacca
ataactgccttaaaaaaattacgccccgccctgccactcatcgcagtactgttgtaattcattaagcattctgccga
catggaagccatcacaaacggcatgatgaacctgaatcgccagcggcatcagcaccttgtcgccttgcgtataa
tatttgcccatggtgaaaacgggggcgaagaagttgtccatattggccacgtttaaatcaaaactggtgaaactc
acccagggattggctgagacgaaaaacatattctcaataaaccctttagggaaataggccaggttttcaccgtaa
cacgccacatcttgcgaatatatgtgtagaaactgccggaaatcgtcgtggtattcactccagagcgatgaaaac
gtttcagtttgctcatggaaaacggtgtaacaagggtgaacactatcccatatcaccagctcaccgtctttcattgc
catacgaaattccggatgagcattcatcaggcgggcaagaatgtgaataaaggccggataaaacttgtgcttatt
tttctttacggtctttaaaaaggccgtaatatccagctgaacggtctggttataggtacattgagcaactgactgaa
atgcctcaaaatgttctttacgatgccattgggatatatcaacggtggtatatccagtgatttttttctccattttagctt
ccttagctcctgaaaatctcgataactcaasaaatacgcccggtagtgatcttatttcattatggtgaaagttggaa
cctcttacgtgccgatcaacgtctcattttcgccaaaagttggcccagggcttcccggtatcaacagggacacca
ggatttatttattctgcgaagtgatcttccgtcacaggtatttattcgcgataagctcatggagcggcgtaaccgtc
gcacaggaaggacagagaaagcgcggatctgggaagtgacggacagaacggtcaggacctggattgggg
aggcggttgccgccgctgctgctgacggtgtgacgttctctgttccggtcacaccacatacgttccgccattcct
atgcgatgcacatgctgtatgccggtataccgctgaaagttctgcaaagcctgatgggacataagtccatcagtt
caacggaagtctacacgaaggtttttgcgctggatgtggctgcccggcaccgggtgcagtttgcgatgccgga
gtctgatgcggttgcgatgctgaaacaattatcctgagaataaatgccttggcctttatatggaaatgtggaactga
gtggatatgctgtttttgtctgttaaacagagaagctggctgttatccactgagaagcgaacgaaacagtcggga
aaatctcccattatcgtagagatccgcattattaatctcaggagcctgtgtagcgtttataggaagtagtgttctgtc
atgatgcctgcaagcggtaacgaaaacgatttgaatatgccttcaggaacaatagaaatcttcgtgcggtgttac
gttgaagtggagcggattatgtcagcaatggacagaacaacctaatgaacacagaaccatgatgtggtctgtcc
ttttacagccagtagtgctcgccgcagtcgagcgacagggcgaagccctcgagctggttgccctcgccgctgg
gctggcggccgtctatggccctgcaaacgcgccagaaacgccgtcgaagccgtgtgcgagacaccgcggc
cggccgccggcgttgtggatacctcgcggaaaacttggccctcactgacagatgaggggcggacgttgacac
ttgaggggccgactcacccggcgcggcgttgacagatgaggggcaggctcgatttcggccggcgacgtgga
gctggccagcctcgcaaatcggcgaaaacgcctgattttacgcgagtttcccacagatgatgtggacaagcctg
gggataagtgccctgcggtattgacacttgaggggcgcgactactgacagatgaggggcgcgatccttgaca
cttgaggggcagagtgctgacagatgaggggcgcacctattgacatttgaggggctgtccacaggcagaaaa
tccagcatttgcaagggtttccgcccgtttttcggccaccgctaacctgtcttttaacctgcttttaaaccaatatttat
aaaccttgtttttaaccagggctgcgccctgtgcgcgtgaccgcgcacgccgaaggggggtgcccccccttct
cgaaccctcccggtcgagtgagcgaggaagcaccagggaacagcacttatatattctgcttacacacgatgcc
tgaaasaacttcccttggggttatccacttatccacggggatatttttataattattttttttatagtttttagatcttcttttt
tagagcgccttgtaggcctttatccatgctggttctagagaaggtgttgtgacaaattgccctttcagtgtgacaaa
tcaccctcaaatgacagtcctgtctgtgacaaattgcccttaaccctgtgacaaattgccctagaagaagctgttt
tttcacaaagttatccctgcttattgactcttttttatttagtgtgacaatctaaaaacttgtcacacttcacatggatctg
tcatggcggaaacagcggttatcaatcacaagaaacgtaaaaatagcccgcgaatcgtccagtcaaacgacct
cactgaggcggcatatagtctctcccgggatcaaaaacgtatgctgtatctgttcgttgaccagatcagaaaatct
gatggcaccctacaggaacatgacggtatctgcgagatccatgttgctaaatatgctgaaatattcggattgacct
ctgcggaagccagtaaggatatacggcaggcattgaagagtttcgcggggaaggaagtggttttttatcgccct
gaagaggatgccggcgatgaaaaaggctatgaatcttttccttggtttatcaaacgtgcgcacagtccatccaga
gggctttacagtgtacatatcaacccatatctcattcccttctttatcgggttacagaaccggtttacgcagtttcgg
cttagtgaaacaaaagaaatcaccaatccgtatgccatgcgtttatacgaatccctgtgtcagtatcgtaagccgg
atggctcaggcatcgtctctctgaaaatcgactggatcatagagcgttaccagctgcctcaaagttaccagcgta
tgcctgacttccgccgccgcttcctgcaggtctgtgttaatgagatcaacagcagaactccaatgcgcctctcata
cattgagaaaaagaaaggccgccagacgactcatatcgtattttccttccgcgatatcacttccatgacgacagg
atagtctgagggttatctgtcacagatttgagggtggttcgtcacatttgttctgacctactgagggtaatttgtcac
agttttgctgtttccttcagcctgcatggattttctcatactttttgaactgtaatttttaaggaagccaaatttgagggc
agtttgtcacagttgatttccttctctttcccttcgtcatgtgacctgatatcgggggttagttcgtcatcattgatgag
ggttgattatcacagtttattactctgaattggctatccgcgtgtgtacctctacctggagtttttcccacggtggata
tttcttcttgcgctgagcgtaagagctatctgacagaacagttcttctttgcttcctcgccagttcgctcgctatgctc
ggttacacggctgcggcgagcgctagtgataataagtgactgaggtatgtgctcttcttatctccttttgtagtgttg
ctcttattttaaacaactttgcggttttttgatgactttgcgattttgttgttgctttgcagtaaattgcaagatttaataaa
aaaacgcaaagcaatgattaaaggatgttcagaatgaaactcatggaaacacttaaccagtgcataaacgctgg
tcatgaaatgacgaaggctatcgccattgcacagtttaatgatgacagcccggaagcgaggaasataacccgg
cgctggagaataggtgaagcagcggatttagttggggtttcttctcaggctatcagagatgccgagaaagcagg
gcgactaccgcacccggatatggaaattcgaggacgggttgagcaacgtgttggttatacaattgaacaaatta
atcatatgcgtgatgtgtttggtacgcgattgcgacgtgctgaagacgtatttccaccggtgatcggggttgctgc
ccataaaggtggcgtttacaaaacctcagtttctgttcatcttgctcaggatctggctctgaaggggctacgtgtttt
gctcgtggaaggtaacgacccccagggaacagcctcaatgtatcacggatgggtaccagatcttcatattcatg
cagaagacactctcctgcctttctatcttggggaaaaggacgatgtcacttatgcaataaagcccacttgctggcc
ggggcttgacattattccttcctgtctggctctgcaccgtattgaaactgagttaatgggcaaatttgatgaaggta
aactgcccaccgatccacacctgatgctccgactggccattgaaactgttgctcatgactatgatgtcatagttatt
gacagcgcgcctaacctgggtatcggcacgattaatgtcgtatgtgctgctgatgtgctgattgttcccacgcctg
ctgagttgtttgactacacctccgcactgcagtttttcgatatgcttcgtgatctgctcaagaacgttgatcttaaag
ggttcgagcctgatgtacgtattttgcttaccaaatacagcaatagcaatggctctcagtccccgtggatggagg
agcaaattcgggatgcctggggaagcatggttctaaaaaatgttgtacgtgaaacggatgaagttggtaaaggt
cagatccggatgagaactgtttttgaacaggccattgatcaacgctcttcaactggtgcctggagaaatgctcttt
ctatttgggaacctgtctgcaatgaaattttcgatcgtctgattaaaccacgctgggagattagataatgaagcgtg
cgcctgttattccaaaacatacgctcaatactcaaccggttgaagatacttcgttatcgacaccagctgccccgat
ggtggattcgttaattgcgcgcgtaggagtaatggctcgcggtaatgccattactttgcctgtatgtggtcgggat
gtgaagtttactcttgaagtgctccggggtgatagtgttgagaagacctctcgggtatggtcaggtaatgaacgt
gaccaggagctgcttactgaggacgcactggatgatctcatcccttcttttctactgactggtcaacagacaccg
gcgttcggtcgaagagtatctggtgtcatagaaattgccgatgggagtcgccgtcgtaaagctgctgcacttacc
gaaagtgattatcgtgttctggttggcgagctggatgatgagcagatggctgcattatccagattgggtaacgatt
atcgcccaacaagtgcttatgaacgtggtcagcgttatgcaagccgattgcagaatgaatttgctggaaatatttc
tgcgctggctgatgcggaaaatatttcacgtaagattattacccgctgtatcaacaccgccaaattgcctaaatca
gttgttgctcttttttctcaccccggtgaactatctgcccggtcaggtgatgcacttcaaaaagcctttacagataaa
gaggaattacttaagcagcaggcatctaaccttcatgagcagaaaaaagctggggtgatatttgaagctgaaga
agttatcactcttttaacttctgtgcttaaaacgtcatctgcatcaagaactagtttaagctcacgacatcagtttgctc
ctggagcgacagtattgtataagggcgataaaatggtgcttaacctggacaggtctcgtgttccaactgagtgtat
agagaaaattgaggccattcttaaggaacttgaaaagccagcaccctgatgcgaccacgttttagtctacgtttat
ctgtctttacttaatgtcctttgttacaggccagaaagcataactggcctgaatattctctctgggcccactgttcca
cttgtatcgtcggtctgataatcagactgggaccacggtcccactcgtatcgtcggtctgattattagtctgggacc
acggtcccactcgtatcgtcggtctgattattagtctcggaccacggtcccactcgtatcgtcggtctgataatca
gactgggaccacggtcccactcgtatcgtcggtctgattattagtctgggaccatggtcccactcgtatcgtcggt
ctgattattagtctgggaccacggtcccactcgtatcgtcggtctgattattagtctggaaccacggtcccactcgt
atcgtcggtctgattattagtctgggaccacggtcccactcgtatcgtcggtctgattattagtctgggaccacgat
cccactcgtgttgtcggtctgattatcggtctgggaccacggtcccacttgtattgtcgatcagactatcagcgtga
gactacgattccatcaatgcctgtcaagggcaagtattgacatgtcgtcgtaacctgtagaacggagtaacctcg
gtgtgcggttgtatgcctgctgtggattgctgctgtgtcctgcttatccacaacattttgcgcacggttatgtggaca
aaatacctggttacccaggccgtgccggcacgttaaccgggctgcatccgatgcaagtgtgtcgctgtcgacg
agctcgcgagctcggacatgaggttgccccgtattcagtgtcgctgatttgtattgtctgaagttgtttttacgttaa
gttgatgcagatcaattaatacgatacctgcgtcataattgattatttgacgtggtttgatggcctccacgcacgttg
tgatatgtagatgataatcattatcactttacgggtcctttccggtgatccgacaggttacggggcggcgacctcg
cgggttttcgctatttatgaaaattttccggtttaaggcgtttccgttcttcttcgtcataacttaatgtttttatttaaaat
accctctgaaaagaaaggaaacgacaggtgctgaaagcgagctttttggcctctgtcgtttcctttctctgtttttgt
ccgtggaatgaacaatggaagtccgagctcatcgctaataacttcgtatagcatacattatacgaagttatattcg
at

The culture medium for inducing an increase in a plasmid copy number and use thereof provided in the present invention are described in detail above. The principle and implementations of the present invention are described herein through specific examples. The description about the foregoing examples is merely provided to help understand the method and core ideas of the present invention. It should be noted that a person skilled in the art may further make several improvements and modifications to the present invention without departing from the principle of the present invention. These improvements and modifications also fall within the protection scope of the claims of the present invention.

What is claimed is:

1. A culture medium for inducing an increase in a plasmid copy number, comprising tryptone, a yeast extract, glucose, sodium chloride, and arabinose.

2. The culture medium for inducing an increase in a plasmid copy number according to claim 1, wherein the glucose has a concentration of from 0.1 to 10 g/L, preferably from 1 to 5 g/L, and more preferably from 0.5 to 2 g/L.

3. The culture medium for inducing an increase in a plasmid copy number according to claim 1, wherein the arabinose has a concentration of from 0.1 to 10 g/L, preferably from 1 to 5 g/L, and more preferably from 0.3 to 0.75 g/L.

4. The culture medium for inducing an increase in a plasmid copy number according to claim 1, wherein the tryptone has a concentration of from 1 to 15 g/L, and preferably from 5 to 10 g/L.

5. The culture medium for inducing an increase in a plasmid copy number according to claim 1, wherein the yeast extract has a concentration of from 1 to 15 g/L, and preferably from 5 to 10 g/L.

6. The culture medium for inducing an increase in a plasmid copy number according to claim 1, wherein the sodium chloride has a concentration of from 1 to 15 g/L, and preferably from 5 to 10 g/L.

7. The culture medium for inducing an increase in a plasmid copy number according to any one of claims 1 to 6, further comprising one or more of magnesium chloride, potassium chloride, ferric chloride, or calcium chloride.

8. The culture medium for inducing an increase in a plasmid copy number according to any one of claims 1 to 7, wherein the plasmid is a plasmid comprising a replication origin oriV.

9. The culture medium for inducing an increase in a plasmid copy number according to claim 8, wherein the plasmid is a single-copy plasmid comprising a replication origin oriV, preferably a pCCIBAC plasmid.

10. A method for inducing an increase in a plasmid copy number, comprising the following steps:
Step (1): transforming Escherichia coli with a plasmid; and
Step (2): inoculating the Escherichia coli obtained in Step (1) into the culture medium according to any one of claims 1 to 9 for culturing under conditions suitable for culture.

11. The method for inducing an increase in a plasmid copy number according to claim 10, wherein in Step (1), the plasmid is transformed into competent Escherichia coli.

12. The method for inducing an increase in a plasmid copy number according to claim 10, wherein in Step (1), the Escherichia coli is EPI300 or EPI400 Escherichia coli.

13. The method for inducing an increase in a plasmid copy number according to claim 10, wherein in Step (2), a culture temperature is from 25 to 37° C., and a culture time is from 4 to 6 h.

14. Use of the culture medium according to any one of claims 1 to 9 in inducing an increase in a plasmid copy number.
